(19) United States
(12) Patent Application Publication
(10) Pub. No.: US 2021/0401418 A1
(43) Pub. Date: Dec. 30, 2021

(54) OCCLUDER WITH SELF-POWERED SENSORS

(71) Applicant: Edwards Lifesciences Corporation, Irvine, CA (US)

(72) Inventors: Lynn T. Dang, Huntington Beach, CA (US); Hao-Chung Yang, Tustin, CA (US); Javier A. Sanguinetti, Irvine, CA (US)

(21) Appl. No.: 17/469,784

(22) Filed: Sep. 8, 2021

Related U.S. Application Data
(63) Continuation of application No. PCT/US2020/019615, filed on Feb. 25, 2020.
(60) Provisional application No. 62/817,199, filed on Mar. 12, 2019.

(51) Int. Cl. A61B 17/00; A61B 5/00; A61B 5/0205

(52) U.S. Cl. CPC A61B 17/0057; A61B 5/686; A61B 5/02055; A61B 5/0004; A61B 2017/0011; A61B 2017/00592; A61B 2017/00632; A61B 2560/0214; A61B 2562/0247; A61B 5/0015

(57) ABSTRACT

Disclosed herein are left atrial appendage (LAA) occluders that include self-powered physiological sensors to monitor physiological parameters of a subject. The sensors can be powered by harvesting energy generated by the patient's body or using wireless power delivery technologies. The disclosed devices can be used to close the LAA and to provide self-powering sensors to wirelessly monitor physiological parameters such as heart rate, pressure, temperature, size of the atrium, and levels of biomarkers such as C-reactive protein (CRP) and B-type natriuretic peptide (BNP) (e.g., using biosensors). In addition to addressing the stroke risk for patients with non-valvular atrial fibrillation, the disclosed devices offer post-surgical connected care that can reduce hospital readmissions, provide superior medical management, and improve patient quality of life.

### CROSS REFERENCE TO RELATED APPLICATIONS

This application is a continuation of International Patent Application No. PCT/US2020/019615, filed Feb. 25, 2020, which claims the benefit of U.S. Patent Application No. 62/817,199, filed Mar. 12, 2019, the entire disclosures which are incorporated by reference for all purposes.

### BACKGROUND

### Description of Related Art

Open heart surgery is associated with a very high incidence of peri-operative atrial fibrillation. In valve repair or replacement, the rate of peri-operate atrial fibrillation is approximately 45%. In patients with non-valvular atrial fibrillation, embolic stroke is thought to occur from thrombi forming in the left atrium, with the left atrial appendage (LAA) being the principal site of thrombus formation. In atrial fibrillation, the heart's upper chambers, or atria, beat irregularly. Pooling of blood flow during atrial fibrillation in the LAA can increase the risk of blood clot formations that could travel to the brain and cause a stroke. Antiarrhythmic drugs and catheter ablation may be effective in symptomatic relief for patients with atrial fibrillation and the prevention of thromboembolic events may be treated using oral anticoagulation (e.g., vitamin K antagonists, VKA).

The LAA is a small, ear-shaped sac in the muscle wall of the left atrium. Among patients that do not have valve disease, the majority of blood clots that occur in the left atrium start in the LAA. In some circumstances, it may be advantageous to occlude or seal off the LAA to reduce a risk of stroke and to reduce or eliminate the need to take blood-thinning medication.

### SUMMARY

This summary is meant to provide some examples and is not intended to be limiting of the scope of the disclosure in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. The description herein relates to systems, assemblies, methods, devices, apparatuses, combinations, etc. that may be utilized for valve repair. Various features and steps as described elsewhere in this disclosure can be included in the examples summarized here.

In some aspects, the present disclosure relates to a device for occluding a cavity inside of a subject. The device comprises a cover or covering (e.g., a membrane, fabric, cloth, polymer layer, etc.) with an outer surface and an inner surface, the cover or covering configured to inhibit passage of blood. The device also comprises a frame (e.g., an expandable frame, etc.) at least partially covered by the cover or covering, the frame configured to support the cover or covering in the cavity to substantially occlude the cavity. The frame can optionally include a plurality of longitudinally extending beams coupled together using pairs of struts. The device also comprises a structure (e.g., an energy harvesting structure, a support structure, a power generator, etc.), which can be coupled to the cover or covering and/or to the frame. The structure is configured to harvest energy from environmental sources within or around the cavity. The device also comprises at least one sensor (e.g., one sensor, two sensors, three sensors, a plurality of sensors, etc.). The at least one sensor(s) can be coupled to the outer surface of the cover or covering. The at least one sensor is configured to receive power from the energy harvested by the structure.

In some embodiments, the at least one sensor is one of a plurality of physiological sensors coupled to the device. In some embodiments, the at least one sensor comprises an absolute pressure measurement sensor.

In some embodiments, the device further comprises circuitry with an electrical connection to the at least one sensor. An antenna can be coupled to the circuitry wherein the circuitry includes a transmitter coupled to the antenna to transmit data acquired by the at least one sensor. The circuitry can be configured to receive power from the energy harvested by the structure. The circuitry can include a battery that is recharged using the received power.

In some embodiments, the structure includes a stack of piezoelectric polymers configured to generate electrical power from mechanical deflections or deformations. In some embodiments, the structure comprises layers of piezoelectric material separated by conductive plates.

In some embodiments, the structure is incorporated into and/or is integral with the frame. The structure can be configured to further generate data related to blood pressure.

In some embodiments, the device further comprises an ultrasound receiver module configured to receive ultrasound transmissions. The ultrasound receiver module can be configured to receive power from an external ultrasound source using ultrasound.

In some embodiments, the device further comprises an ultrasound transmission module configured to transmit data to the external ultrasound source using ultrasound.

In some embodiments, the structure is affixed to the outer surface of the cover or covering. The cover or covering can form a dome structure and the structure extends over a center of the dome.

A patient monitoring system herein can comprise an occlusion device with a cover or covering (e.g., a membrane, fabric, cloth, polymer layer, etc.) and a frame (e.g., an expandable frame, etc.) configured to occlude a cavity inside of a subject. The device includes one or more sensors (which can be associated with and/or part of the cover or covering, frame, etc.) that receive electrical power from a power generator associated with the frame and/or cover. The one or more sensors can include a plurality of physiological sensors. The power generator can be configured to generate electrical power in response to deformation of the frame and/or cover.

The occlusion device can further include an antenna in communication with the one or more sensors to transmit data acquired using the one or more sensors.

In some embodiments, the system further includes an external local monitor configured to receive data transmitted from the occlusion device, the external local monitor including a data display configured to display data acquired with the one or more sensors of the occlusion device.

In some embodiments, the occlusion device further includes a receiver to receive wireless transmission from the external local monitor.

In some embodiments, the system further comprises a remote monitor configured to receive data from the external local monitor to enable monitoring of data acquired with the one or more sensors remotely.

In some embodiments, the system comprises a secondary local monitor configured to provide an interface for interacting with the data from the one or more sensors of the occlusion device.

In some aspects, the present disclosure relates to a device for occluding a left atrial appendage (LAA) of a subject. The device includes a membrane with an outer surface and an inner surface, the membrane configured to inhibit passage of blood. The device also includes an expandable frame at least partially covered by the membrane, the expandable frame configured to support the membrane in the LAA to substantially occlude the LAA. The device also includes a support structure associated with the membrane and/or the expandable frame, the support structure configured to harvest energy from environmental sources within the LAA. The device also includes a plurality of physiological sensors coupled to the outer surface of the membrane, the plurality of physiological sensors configured to receive power from the energy harvested by the support structure. In some aspects, the present disclosure relates to a similar device, but for occluding another cavity in the body other than the LAA, such as another appendage, bulge, or aneurysm.

In some embodiments, the device further includes circuitry with electrical connections to the plurality of physiological sensors. In some embodiments, the device further includes an antenna coupled to the circuitry wherein the circuitry includes a transmitter coupled to the antenna to transmit data acquired by one or more of the plurality of physiological sensors. In some embodiments, the circuitry receives power from the energy harvested by the support structure. In some embodiments, the circuitry includes a battery that is recharged using the received power.

In some embodiments, the support structure includes a stack of piezoelectric polymers configured to generate electrical power from mechanical deflections or deformations. In some embodiments, the support structure is incorporated into the expandable frame. In some embodiments, the support structure is incorporated into the membrane. In some embodiments, the support structure further generates data related to blood pressure. In some embodiments, the support structure comprises layers of piezoelectric material separated by conductive plates. In some embodiments, the plurality of physiological sensors includes an absolute pressure measurement sensor.

In some embodiments, the device further includes an ultrasound receiver module configured to receive ultrasound transmissions. In some embodiments, the ultrasound receiver module is configured to receive power from an external ultrasound source using ultrasound. In some embodiments, the device further includes an ultrasound transmission module configured to transmit data to the external ultrasound source using ultrasound.

In some embodiments, the expandable frame includes a plurality of longitudinally extending beams coupled together using pairs of struts. In some embodiments, the support structure is affixed to the outer surface of the membrane. In some embodiments, the membrane forms a dome structure and the support structure extends over a center of the dome.

In some aspects, a patient monitoring system is disclosed that includes a left atrial appendage (LAA) occlusion device with a membrane and an expandable frame configured to occlude an LAA of a subject, the membrane including a plurality of sensors that receive electrical power from a power generator associated with the expandable frame or the membrane, the power generator configured to generate electrical power in response to deformation of the expandable frame or the membrane, the LAA occlusion device further including an antenna in communication with the plurality of sensors to transmit data acquired with the plurality of sensors. The system also includes an external local monitor configured to receive data transmitted from the LAA occlusion device, the external local monitor including a data display configured to display data acquired with the plurality of sensors of the LAA occlusion device. In some aspects, the present disclosure relates to a similar device, but for occluding another cavity in the body other than the LAA, such as another appendage, bulge, or aneurysm.

In some embodiments, the occlusion device further includes a receiver to receive wireless transmission from the external local monitor. In some embodiments, the system further includes a remote monitor configured to receive data from the external local monitor to enable monitoring of data acquired with the plurality of sensors remotely. In some embodiments, the system further includes a secondary local monitor configured to provide an interface for interacting with the data from the plurality of sensors of the occlusion device.

### BRIEF DESCRIPTION OF THE DRAWINGS

Various embodiments are depicted in the accompanying drawings for illustrative purposes, and should in no way be interpreted as limiting the scope of the disclosure. In addition, various features of different disclosed embodiments can be combined to form additional embodiments, which are part of this disclosure. Throughout the drawings, reference numbers may be reused to indicate correspondence between reference elements.

FIGS. 1A and 1B illustrate an example occluder or left atrial appendage (LAA) occluder that includes a membrane component configured to inhibit passage of blood and an expandable frame having a cupped occlusive component at least partially covered with the membrane component, the membrane including a plurality of self-powered sensors affixed thereto.

FIGS. 1C and 1D illustrate the occluder or LAA occluder of FIGS. 1A and 1B being implanted in the LAA (or another cavity) of a subject.

FIG. 1E illustrates a top view of an example support structure for use in a percutaneous occluder.

FIG. 1F illustrates a top view of an example support structure for use in a surgical occluder.

FIG. 2 illustrates an example occlusion device that includes self-powered sensors that receive power from energy harvested by a support structure.

FIG. 3 illustrates an example system for monitoring the on-going health of a patient using sensors of occlusion devices described herein.

FIG. 4 illustrates a diagram of an example electronic sensor module incorporated with occlusion devices described herein.

FIG. 5 illustrates a block diagram of an example external local monitor system configured to communicate with a sensor module.

FIG. 6 illustrates an example power and/or data communication system that utilizes ultrasound to transmit power to an occlusion device.

FIG. 7 illustrates an example of an external coil device that can be used for coupling with an occlusion device.

### DETAILED DESCRIPTION

The headings provided herein are for convenience only and do not necessarily affect the scope or meaning of any of the claimed embodiments.

### Overview

Evidence points to the left atrial appendage (LAA) as the primary origin of thrombus formation particularly in the presence of non-valvular atrial fibrillation (AF). Because a major risk of non-valvular AF is an ischemic stroke, preventing thrombus formation in the LAA can be beneficial. Stroke prevention in patients with non-valvular AF may involve the use of oral anticoagulants or antiplatelet agents or LAA occlusion or exclusion (e.g., LAA closure).

LAA or other appendage or aneurysm closure devices can be designed and configured in a variety of ways. In some applications, closure devices can be at least one of an occluder and/or a clamp.

Occluders can be designed and configured to fill the LAA or other appendage, bulge, or aneurysm to close the cavity to thrombus formation and/or to prevent thrombi in the cavity from escaping into the blood stream. Occluders can be configure in a variety of ways. In some applications, occluders comprise stents, covered stents, nitinol covered half stents, braided disks, etc. Occluders can be configured to be delivered transvascularly with a delivery catheter navigating through vasculature to the cavity and to be implanted in the cavity to occlude it.

Clamps can be configured to be applied to the appendage, bulge, or aneurysm in a way that forces (e.g., pulls, pushes, etc.) different portions of the tissue together to close the cavity. In some applications, a clamp device is applied externally to the appendage or aneurysm during surgery. If a surgeon leaves a “neck” portion of the appendage, this can remain susceptible to thrombus formation.

LAA closure procedures can include LAA exclusion with sutures on the epicardial or endocardial surface and LAA excision through staples or removal and over-sew. Percutaneous approaches for LAA occlusion include obstruction of the LAA orifice with an occlusion device or percutaneous suture ligation using an endocardial/epicardial approach.

Because typical patients that would receive an occluder device suffer from non-valvular AF and may also suffer from other cardiac-related issues, it may be beneficial to monitor their physiological state even after being discharged from the hospital or extended care facility. In addition, patients who receive an occluder may suffer from procedure-related complications. Examples of such complications include stroke, pericardial effusion, device embolism, and death. It may be that the patient is no longer in a hospital or extended care facility, and therefore complications that arise may require reentry into the care facility, potentially adding significant cost to the overall patient treatment. Furthermore, increased health risks may result from the patient delaying return to the hospital due to failure to recognize the complications until they manifest through perceivable symptoms that the patient interprets as requiring hospital care.

Accordingly, disclosed herein are occluder devices (e.g., LAA occluder devices, etc.) that include self-powered physiological sensors. These sensors can be used to monitor various physiological parameters of a subject and can be powered by harvesting energy generated by the patient's body and/or using wireless power delivery technologies. The disclosed devices can be used to close the LAA or another appendage, bulge, or aneurysm to reduce stroke in patients with non-valvular AF and to provide self-powering sensors to wirelessly monitor a variety of physiological parameters. These parameters can include, for example and without limitation, heart rate, pressure, temperature, size of the atrium, and levels of biomarkers such as C-reactive protein (CRP) and B-type natriuretic peptide (BNP) (e.g., using biosensors). In addition to addressing the stroke risk for patients with non-valvular AF, the disclosed devices offer post-surgical connected care that can reduce hospital readmissions, provide superior medical management, and improve patient quality of life.

In some embodiments, the disclosed devices can be dome-shaped with sensors attached to the outer side of the dome. The disclosed devices can include piezoelectric material and/or an energy storage device (e.g., batteries such as solid-state batteries, capacitors, or the like) for energy harvesting and supply. The piezoelectric material can be incorporated into a frame of the device and/or into the dome. The disclosed devices can include one or more antennas for data communication (e.g., where data can be measurements and other information acquired by the sensors attached to the device). Data can be transmitted wirelessly using wireless protocols or technologies such as Wi-Fi, BLUETOOTH®, RFID, near field communication (NFC), and the like. Wireless connectivity allows a health care provider to access data about a patient using a remote device (e.g., a device outside of the patient's body and/or remote from the patient). Additionally, patients may be able to access basic data, heart health info, and other physiological parameters using a personal device (e.g., through a smartphone application).

The disclosed occluder devices and LAA occluder devices also enable post-operative monitoring of subjects, including possibly in an environment outside of the relevant hospital or care facility. Certain embodiments disclosed herein provide an occluder device/system including integrated sensing capability for sensing one or more conditions of the occluder device and/or heart of a patient. The device can be configured to wirelessly communicate sensed parameters (e.g., critical patient issues) from the sensor system in the device to a local or remote wireless receiver device. In some embodiments, the local or remote device can be carried by the patient. The receiver can be configured to communicate information associated with the received sensor information to a care provider system, such as to a remote hospital or care facility monitoring system.

Physiological parameters that can be tracked by sensor-enabled occluders can include arrhythmia, blood pressure, cardiac output (e.g., as measured by an echo sensor, induction, ballistocardiogram, or the like), temperature, glucose levels, and/or other parameter(s). Furthermore, occluder devices disclosed herein can incorporate any desired or practical types of sensors, such as strain gauges, pressure sensors, optical sensors, audio sensors, position sensors, acceleration sensors, or other type(s) of sensors. Integrated implant sensors can advantageously be configured to generate electrical signals that can be wirelessly transmitted to a receiver device (e.g., box) disposed outside the patient's body. In certain embodiments, the receiver device is configured to forward information based at least in part on the signals to a remote care giver system/entity.

In certain embodiments, sensor devices associated with occlusion devices can sense pressure and/or electrical activity. Electrical activity sensor(s) can provide information used to detect arrhythmia or other conditions. Pressure sensors integrated in devices disclosed herein can include microelectromechanical (MEMS) devices (e.g., accelerometer), which can be integrated in the device frame, for example. In certain embodiments, two or more sensors can be utilized.

Sensors and/or transmitters integrated in devices disclosed herein can be powered using the patient's body movement. For example, patient movement (e.g., through beating of the heart) can be used to generate power, such as by using one or more piezoelectric MEMS devices (e.g., strain gauge, accelerometer). Certain embodiments disclosed herein include sensors with energy harvesting feature(s) for generating power for sensor operation and/or data transmission from environmental conditions. For example, an occluder device or LAA occluder device can include a piezoelectric sensor or device, or other passive power generator, wherein the piezoelectric sensor/device is configured to generate an electrical signal in response to fluid pressure or other external stimulus. The piezoelectric sensor can advantageously be integrated with one or more structural features of an occluder device, such as a frame, a membrane, or the like. The power generated by the sensor may be sufficient to power the functionality of the physiological sensor, or may serve to supplement another power source, which can be internal or external.

In some embodiments, in addition to harvesting power from the patient, the disclosed devices can use a battery, such as a lithium ion or magnesium-based battery. For example, a battery can use a piece of magnesium as a cathode in at least partial contact with body fluid(s) (e.g., blood), which may degrade as it generates electrical power. In certain embodiments, an external power source configured to provide power through ultrasound, induction, radio frequency (RF) transmission, or other type of wireless power transmission can be used. In certain embodiments, an internal rechargeable battery or capacitor (e.g., supercapacitor) can be used for power storage between charging. Such a power transmitter can be integrated with an external data receiver. In certain embodiments, a portion of the frame of the implant/sensor device can be used as an antenna for power transmission.

In certain embodiments, sensors integrated with an occluder device can be configured to run substantially continuously. Alternatively, the sensor(s) can run only for predetermined intervals, which may provide power savings compared to continuous operation. In certain embodiments, controller logic is integrated with the occluder device for determining timing and/or duration of operation based on measured conditions. In certain embodiments, the sensor(s) operates only when wirelessly coupled with an external data/power communication device. In embodiments in which the sensor(s) collect data even when the device is not coupled to an external device, it may be necessary or desirable for the implant/sensor to include data storage, such as flash memory, memristor(s), or other low-power memory, for storing collected data in interim periods of time.

Certain embodiments operate in connection with an external power/data transfer device, which can advantageously be small enough to be carried with the patient (e.g., continuously), such as by using a chest strap, or the like. In certain embodiments, the external device comprises a patch or band with one or more antennae for input/output (I/O) and/or power; remaining circuitry can be contained in a separate box or device. In certain embodiments, the external device comprises an arm-strap fitted device, a chest-strap fitted device, or a device that can fit in the patient's pocket. BLUETOOTH®, near-field communication (NFC), or other low-power technology or protocol can be used to connect the external device and/or implant/sensor to a smartphone or other computing device to transmit data to a hospital or other data aggregator.

Certain embodiments disclosed herein provide a laminated piezoelectric-polymer electricity generator integrated onto occluder devices for harvesting energy from blood flow-induced vibrations and movement of support frames or a membrane to power electronic implantable medical devices, such as blood-pressure sensors, blood glucose meters, pacemakers, electrocardiography sensors (ECG), temperature sensors, pulse oximetry sensors, and the like. Sensor devices disclosed herein can be self-powered, such as through energy harvesting means and/or battery power.

### Example Occluder Devices

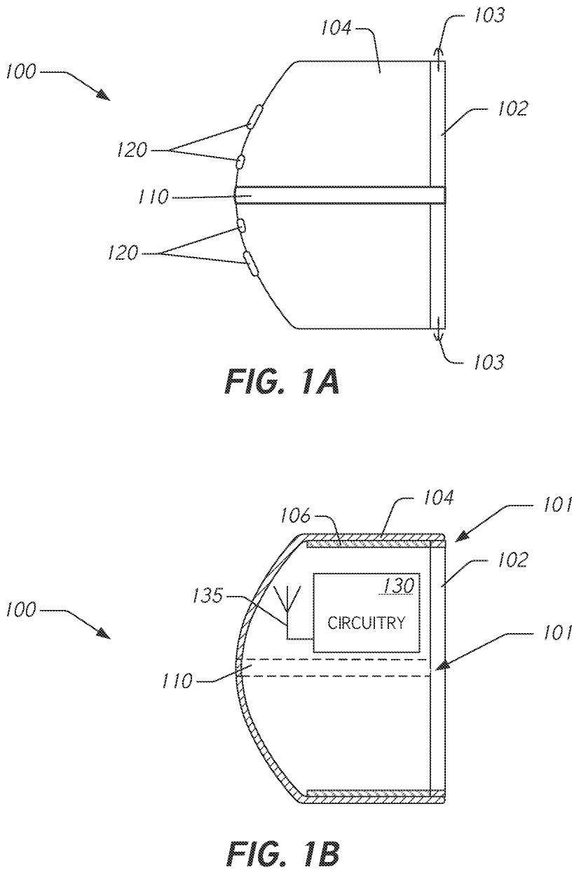

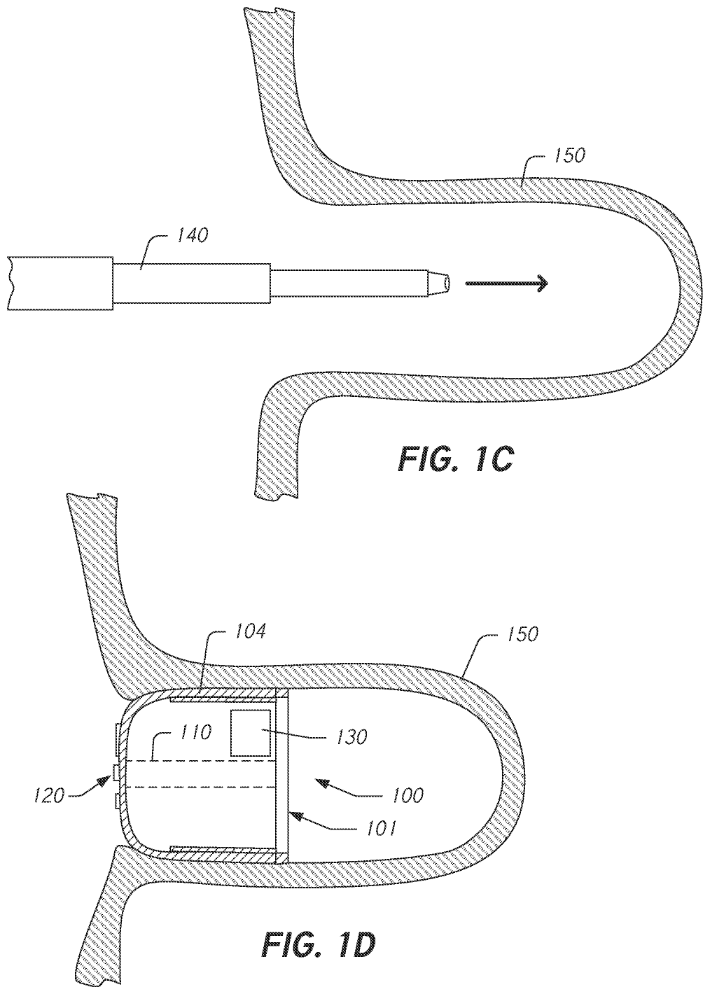

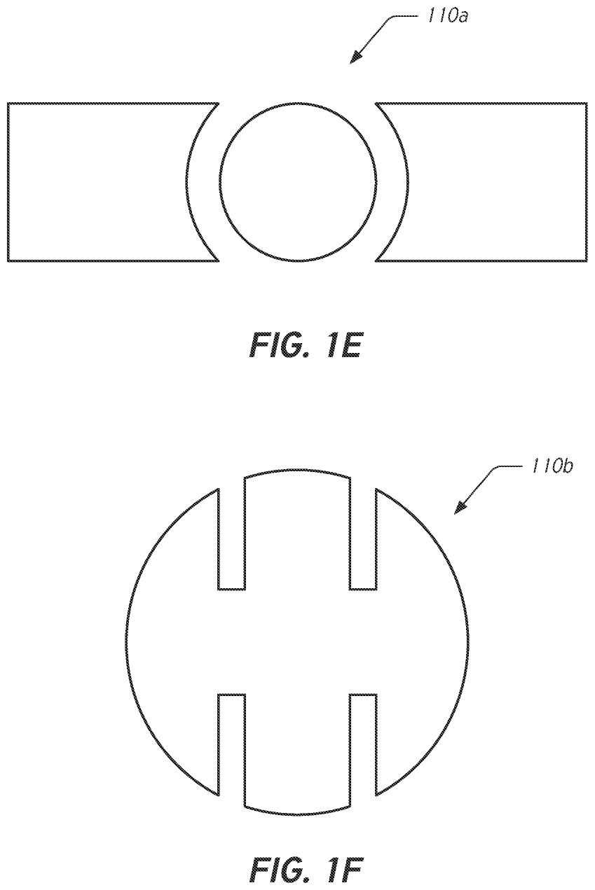

FIGS. 1A and 1B illustrate an example occluder 100, sometimes referred to or depicted herein as an LAA occluder, but it should be understood that this is representative of occluders that can be used in locations other than the LAA. Occluder 100 includes a membrane component 104 configured to inhibit passage of blood and an expandable frame 101 having a cupped occlusive component at least partially covered with the membrane component 104, with one or more anchors 103. The occluder 100 includes a frame base 102, frame supports 106, and a dome or membrane 104 with sensors 120 attached to the outer side of the membrane 104. The occluder 100 also includes a support structure 110 that can extend from the frame base 102 over the membrane 104 that can be used to harvest energy to power the sensors 120, circuitry 130, and antenna 135. The frame base 102 and the frame supports 106 can be collectively referred to as a frame 101. In some embodiments, the support structure 110 also forms part of the frame 101. In some embodiments, the support structure 110 is incorporated into the membrane 104.

In some embodiments, the occluder 100 includes a nitinol cage (e.g., the frame 101) enclosed in an ePTFE membrane (e.g., the membrane 104). In certain embodiments, the LAA occluder 100 is designed to be inserted entirely into the LAA and can include anchors 103 for attachment to the interior wall of the LAA. In some embodiments, the LAA occluder 100 includes a wire cage (e.g., the frame 101) partially covered by an ePTFE membrane (e.g., the membrane 104). In some embodiments, the occluder 100 has a self-expanding nitinol frame (e.g., the frame 101) with fixation barbs 103 and a permeable polyester fabric cover (e.g., the membrane 104). In some embodiments, the frame 101 includes a plurality of discrete frame segments coupled with at least one ring member to form a frame structure. A tissue growth member (e.g., the membrane 104) is coupled with the plurality of discrete frame segments to define a substantially convex surface and a substantially concave surface.

The occluder 100 can be inserted minimally invasively. The frame 101 can be considered a securement or retention member and the membrane 104 can be configured to substantially prevent blood from at least one of entering and exiting the left atrial appendage. As an example, the membrane 104 can be made of a biocompatible mesh material. The frame 101 provides for attachment to the appendage wall as well as act as a support or retention member for the membrane 104.

The expandable frame 101 can be constructed from wires, for example fatigue resistant wires, that have elastic properties. In some embodiments, expandable frame 101 is constructed of wires that have elastic properties that allow for the expandable frame 101 to be collapsed for catheter-based delivery or thoracoscopic delivery, and then self-expand to the desired configuration once positioned in a cavity.

The material for the frame 101 can be selected for its biocompatibility, including its anti-thrombogenic capacity, its shape-recovery capabilities and super-elasticity. The material for the frame 101 may comprise a metal or a metal alloy. The material for the frame 101 can be a spring wire, a shape memory alloy wire or a super-elastic alloy wire. Any material can be used that has biocompatible characteristics and is strong, flexible, and resilient. The material can be, for example, nitinol (NiTi), L605 steel, stainless steel, or any other biocompatible wire. The material can also be of a drawn-filled type of nitinol containing a different metal at the core. The super-elastic properties of nitinol make it a useful material for this application. Nitinol wire can be heat set into a desired shape. Stainless steel wire is an alternative material. It can be plastically deformed into a desired shape. Other shape memory or plastically deformable materials can be suitable in this application.

The membrane 104 can be configured to substantially or completely prevent blood from entering and/or exiting the cavity. The membrane 104 can be configured as a tissue growth member, or a surface which facilitates rather than impedes tissue growth. The membrane 104 can include a porous member configured to promote tissue in-growth thereon. The membrane 104 can be a polymeric material, such as foam or other materials. The membrane 104 can exhibit a cup-like shape having an outer (or convex) surface and an inner (or concave) surface. The membrane 104 can be sized and configured to be in direct contact with tissue within the LAA or other cavity.

The frame 101 or the various structures of the frame 101 are configured to assist in expanding the membrane 104 and to assist in collapsing the membrane 104 for delivery through an associated catheter or other medical device. The frame 101 assists in collapsing the membrane 104 (such as during a loading procedure) to a size wherein the occluder 100 fits within the lumen of a catheter and may be displaced therethrough without damaging the membrane 104. Further, when deploying the collapsed membrane 104 from a catheter, the frame 101 is configured to self-expand to assist in opening the membrane 104 so that a large portion of the outer surface of the membrane 104 is in direct contact with the tissue of the cavity or appendage.

The membrane 104 can include a fabric material that facilitates tissue growth over the occluder or LAA occluder 100. The fabric material can be any suitable shape that fits within or over the frame 101. For example, the fabric material may be a sheet, a plurality of sheets, a membrane or a random shape that fits over at least a portion of the frame 101 or, in some embodiments, fills up at least a portion of the inside of the frame 101. The fabric material can be any suitable material that promotes and/or facilitates tissue growth so that tissue of the subject can grow in and around the occluder 100. For example, the fabric material can be any suitable polyester fibers, such as Dacron®. Alternatively, the fabric material can be made of a biodegradable and/or biocompatible material such as expanded polytetrafluoroethylene (ePTFE), Teflon®, felt, Gortex® (a PTFE material), silicone, urethane, metal fibers, other polymers, such as polypropylene, or combinations thereof. The fabric material can be impermeable to fluid, such as blood or body fluid. In some embodiments, the material of the membrane 104 can include a porous foam material.

The occluder 100 provides a frame 101 that is compliant enough to conform to a wide variety of LAA or other appendage or cavity geometries and sizes. Some embodiments of the occluder 100 provide a left atrial appendage occlusion device frame that provides firm, secure anchoring with significantly reduced clinical sequela from piercing or without traumatic piercing of the left atrial appendage tissue or other tissue. Some embodiments provide a membrane component 104 configured to inhibit the passage of blood through the membrane 104. For example, the membrane 104 can be configured to substantially occlude the flow of blood through the membrane 104. Some embodiments provide a membrane or dome 104 that is configured to induce rapid tissue ingrowth and promptly or immediately occludes the passage of blood through the membrane.

In some embodiments, one or more anchors 103 (e.g., barbs) contact the wall or body of the appendage or cavity. In some embodiments the point of contact between the anchors 103 is the endocardial surface within the appendage or cavity. While in some embodiments one or more anchors 103 penetrate into the endocardial surface of the appendage or cavity, in some other embodiments, there is no penetration of the endocardial surface. In some embodiments, some anchors 103 penetrate the endocardial surface while other anchors 103 do not penetrate the endocardial surface. In some embodiments, some barbs 103 penetrate the endocardial surface while other barbs 103 do not penetrate the endocardial surface. In some embodiments, one or more anchors 103 contact trabeculation of the endocardial surface.

In some embodiments, one or more anchors 103 are formed from portions of the lengths of wires of the frame 101. In some embodiments, two or more, three or more, four or more, five or more, six or more, seven or more, eight or more, or nine or more anchors stabilize and/or secure the occluder 100.

In some embodiments, the anchors 103 can be elements that include scalloped edges that are configured to anchor the occluder to the walls of the cavity (e.g., to anchor an LAA occluder to inner walls of the LAA) and to reduce or eliminate the risk of penetration or perforation of the walls. In such embodiments, the anchors 103 do not distend the appendage or cavity and do not have any barbs, hooks, or loops of wire that might cause the anchors 103 to have a sharp end, where sharp ends are prone to perforate the inner walls of the appendage or cavity. Scalloped edges can substantially resemble or resemble a wave formation, such as a sinusoidal shape.

The support structure 110 can be configured to harvest energy by converting mechanical movement into electrical energy. The mechanical movement can cause movement of the frame 101 and/or the membrane 104 in which the piezoelectric material is incorporated. For example, the support structure 110 can be a laminated piezoelectric polymer that generates electricity from mechanical movement resulting from blood flow-induced vibrations and movement. The support structure 110 can be a stack of piezoelectric polymers configured to generate electrical power from mechanical deflections or deformations. The support structure 110 can be any suitable laminate or composite material with energy harvesting properties. In some embodiments, the support structure 110 can be made at least partially of a ceramic. The support structure 110 can be pliable and relatively thin. By straining the piezoelectric elements of the support structure 110 (e.g., through direct piezoelectric effect), the movement of the support structure 110 can generate charge on the surface of the piezoelectric polymer. The resulting capacitive buildup in the polymer can provide a voltage source that can be used, for example, to trickle-charge a battery (which can be part of the circuitry 130), to provide power for data communication (e.g., using the antenna 135), and/or to power the sensors 120, such as blood pressure sensors, blood glucose meters, pacemakers, and/or other devices. In some embodiments, the support structure 110 is also used as a pressure sensor and may be used to measure blood pressure and/or heart rate.

In some embodiments, the support structure 110 is a multi-layered piezoelectric-polymer generator. This electricity generator can be fabricated using a piezoelectric polymer, which may be desirable due to the relatively high piezoelectricity, flexibility, and/or biocompatibility that can be associated with such structures. Unlike piezoelectric ceramics, in which the crystal structure of the material may generally produce electrical energy, piezoelectric polymers can utilize intertwined long-chain molecules to attract and repel each other when an electric field is applied thereto. Furthermore, compared to piezoelectric ceramics, piezoelectric polymers can provide acoustic impedances closer to that of water and/or human tissues, and can have relatively higher voltage constants. For piezoelectric polymers, not only can relatively high sensitivity be an attractive feature for copolymers, but piezoelectric polymers can also crystallize from the melt or from solution in a polar phase. Therefore, it is possible to fabricate such devices in different shapes (e.g., curved surfaces), and pole the copolymer without prior stretching (e.g., reduced fabrication time).

The support structure 110 can comprise layers of piezoelectric material separated by conductive (e.g., metal) plates. The support structure 110 can comprise any suitable piezoelectric material, such as piezoelectric fiber composites, piezoelectric films, or piezoelectric ceramics. In certain embodiments, it may be desirable to use flexible piezoelectric elements, such as, for example, flexible piezoelectric fiber composite elements, which can be configured to generate an electrical charge when they are bent or flexed. The piezoelectric elements can be disposed in electrical contact with electrodes that conduct the electrical energy to the circuitry 130, antenna 135, and/or sensors 120 for immediate use or to a battery or capacitor (e.g., within the circuitry 130) for storage for later use.

Certain embodiments disclosed herein provide relatively small, flexible, multi-layered piezoelectric-polymer devices incorporated into the support structure 110 of the occluder 100 to generate reliable, long-term electricity. Although primarily described as being incorporated into the support structure 110, these piezoelectric-polymer devices can additionally or alternatively be included in the frame base 102, the frame supports 106, and/or the membrane 104. Such piezoelectric energy generators can harvest energy not only from movement-induced vibrations of support frames, but also flow-induced vibrations, such as Kármán vortices.

In some embodiments, the support structure 110 can be used to at least partially cover the membrane 104 so that the membrane 104 hosts the piezoelectric material. In such embodiments, the portion of the support structure 110 that comprises the piezoelectric material is attached to or part of the membrane 104. This may be beneficial in a variety of implementations. For example, strain on the frame 101 may exceed the endurance capabilities of the piezoelectric material in certain instances. In such instances, it may be beneficial to associate the energy-harvesting piezoelectric material with the membrane 104 in addition to or instead of with the frame 101.

Powering sensors and other circuitry of the occluder or LAA occluder 100 with the body's own energy according to embodiments disclosed herein can provide one or more advantages. For example, self-powering can reduce or eliminate the need for additional batteries or other power sources, which may require replacement, as well as external power sources, which may require cable or other attachments. With integrated power-generation functionality, sensor devices can advantageously allow for smaller-scale devices, which can improve implantability prospects. For example, use of a relatively small piezo-polymer electricity generator in place of a larger battery power source can reduce device size, thereby providing more space for diagnostic features and/or wireless communication components, such as BLUETOOTH® and radio-frequency identification (RFID) controllers, antennas, and the like.

In some embodiments, the occluder 100 is configured to be secured within a LAA of a subject to occlude the LAA. In some embodiments, occluder 100 is configured to be secured within another cavity of a subject to occlude the cavity. The shape of the occluder 100 advantageously allows for circuitry 130 and an antenna 135 to be housed within the occluder 100. The circuitry 130 can include a battery that can be used to power the sensors 120 and communication using the antenna 135. Power harvested from the support structure 110 can be used to recharge the battery and/or to provide power directly to the sensors 120, the circuitry 130, and/or the antenna 135. The piezoelectric material that harvests energy from the support structure 110 can be part of the frame 101 and/or part of the membrane 104.

In certain embodiments, the circuitry 130 is configured to perform some amount of signal processing for signal transmission, such as signal filtering, amplification, mixing, and/or the like. In certain embodiments, the circuitry 130 includes one or more processors, data storage devices, data communication buses, and/or the like.

The antenna 135 can include any suitable antenna or combination of electromagnetic emitters and receivers to send and receive electromagnetic signals. The antenna 135 can be configured to communicate using radio frequency signals and may be configured for a variety of wireless communication protocols (e.g., WiFi, BLUETOOTH®, NFC, etc.) In some embodiments, the antenna 135 includes antenna coils for data and/or power transfer between sensor-integrated devices and external monitor devices and can have any desirable or suitable configuration. Near-field communication can involve the use of two parallel-aligned coil loops that are magnetically coupled, one being the transmitter and the other being an antenna with current running through to introduce a magnetic field. To be able to surpass attenuation from the surrounding tissue and fluid within the patient anatomy when the sensor device is implanted in a patient, it may be desirable for the current through the antenna to be run at relatively lower frequencies, which may generally require the use of relatively larger diameter coils. In certain embodiments, the antenna coil may be wrapped at least partially around a core form or volume (e.g., magnetic iron/ferrite core or air core) to help improve coupling. For use in occluders, it may be desirable or advantageous for a ferrite-wrapped coil to be hermetically sealed in a biocompatible casing to prevent exposure to surrounding tissue(s).

The occluder 100 includes one or more sensors 120. The one or more sensors 120 can be configured to provide a response indicative of one or more physiological parameters of a patient, such as one or more parameters associated with the function or integration of the occluder 100 and the associated heart. The sensor(s) 120 can comprise any suitable or desirable sensor(s) for providing signals relating to physiological parameters or conditions associated with occluder 100. In view of the integrated sensor(s) 120, the occluder 100 can advantageously provide sensor capability without the necessity of a separate, stand-alone device that requires a separate procedure to implant.

In certain embodiments, the sensor(s) 120 includes a pressure sensor, such as a pulmonary artery pressure (PAP) measurement device. The sensor(s) 120 can additionally or alternatively comprise one or more optical sensors, piezoelectric sensors, electromagnetic sensors, strain sensors/gauges, accelerometers, gyroscopes, and/or other types of sensors, which can be positioned in a patient to sense one or more parameters relevant to the function of the occluder 100. Sensor signals can be used to track arrhythmia, blood pressure, cardiac output (e.g., as measured by an echo sensor), induction or ballistocardiogram. In an embodiment, the sensor(s) 120 comprises a MEMS pressure sensor, which can be either capacitive or piezoresistive in nature, wherein the sensor is coupled with an application-specific integrated circuit (ASIC) microcontroller. The sensor(s) 120 can be attached to a polyimide flexible circuit substrate and can be further accompanied with one or more discrete electronic components, such as tuning capacitors or the like. In certain embodiments, the sensor(s) 120 comprises one or more electrodes for detecting electrical impulses originating in the heart.

The sensors 120 can include, for example, a strain gauge, which can be attached to, or embedded within, the support structure 110 and/or the frame supports 106. For example, the strain gauge can be attached to, or etched in, the support structure 110 and/or the frame supports 106 which can comprise a plastic (e.g., PET) band. The strain gauge can comprise an electrical conductor that has electrical conductance properties that depend at least in part on the geometry of the conductor; when the support structure 110 deflects in a way as to present tension on the strain gauge, the electrical conductor of the strain gauge can become stretched, thereby becoming relatively narrower and/or longer, which can increase the electrical resistance of the conductor end-to-end. Alternatively, when the support structure 110 deflects in a way as to result in compression of the strain gauge, the electrical conductor of the strain gauge can experience increased thickness, which can decrease the electrical resistance of the conductor end-to-end. The electrical resistance of the strain gauge can therefore be measured, and the amount of deflection or induced stress on the commissure post can be inferred based on such measurement. In certain embodiments, the strain gauge can comprise a conductive channel configured in a zig-zag-type pattern of parallel lines such that a stress in the direction of the orientation of the parallel lines results in a measurable change in resistance over the effective length of the conductive lines.

Any of the elements of the occlusion device 100, including the frame 101 and the membrane 104, may have anti-coagulant coating or a coating to promote endothelial cell growth in order to aid in the prevention of clot formation. The anti-coagulant coating may include heparin, an albumin-binding coating, phosphorylcholine, poly-D,L-lactic acid, prostaglandin, dextran sulfate, or other peptide suitable for coagulation prevention. The coating to promote endothelial cell growth may include pyrolytic carbon, a cryoprecipitate-based coating, autologous fibrin meshwork, elastin-based polypeptides, fibronectin, collagen IV, a fibronectin-collagen IV combination, extracellular matrix proteins and peptides, plasma polymerized coating or other suitable material to encourage growth of endothelial cells on the sheet.

FIGS. 1C and 1D illustrate the occluder 100 being implanted in the LAA 150 or another cavity of a subject. As illustrated in FIG. 1C, to implant the occluder 100, a catheter 140 is threaded through the vasculature and into the heart to deliver the occluder 100 to the LAA 150 or other cavity. As illustrated in FIG. 1D, when the occluder 100 is properly positioned within the LAA 150 the occluder 100 forms a seal with the wall of the LAA 150 to prevent or impede emboli or blood clots from passing back into the blood stream.

The occluder 100 can be introduced via catheter 140 through the femoral vein by transseptal passage. Transesophageal echocardiography (TEE) guiding or intracardiac echocardiography (ICE) can be utilized during the implantation procedure. The occluder 100 can be positioned so that it does or does not protrude beyond the LAA ostium. The occluder 100 can be positioned so that it covers the entire ostium with no or minimal residual flow. The occluder 100 initially becomes coated by fibrin and subsequently covered by endothelial cells forming an endocardial lining, which consequently excludes the occluder 100 from circulating blood. In some embodiments, the catheter 140 can be a pigtail catheter that is advanced into the LAA 150, where the catheter 140 includes a sheath that is advanced over the pigtail into the LAA 150. The pigtail catheter advantageously decreases the probability of LAA or cavity perforation. The preloaded delivery catheter 140 can be advanced into the tip of the access sheath and can be deployed by a gentle retraction of the sheath, in such embodiments.

In some embodiments, the catheter 140 includes a plunger that is slidably disposed within an inner lumen of the delivery catheter 140 and serves to apply axial force in a distal direction on the collapsed occluder 100 disposed within the delivery catheter 140 so as to force the occluder 100 from the delivery catheter 140 and deploy it. The occluder 100 can be guided into the LAA 150 by use of an appropriate guidewire or guiding member.

FIG. 1D illustrates the occluder 100 in a deployed state within the LAA or cavity 150. The frame 101 and membrane 104 are in substantial sealing contact with the inside surface of the LAA 150. The frame 101 has expanded to contact the inside surface of the LAA 150, to secure the occluder 100 thereto, and to maintain the occluder 100 in a substantially perpendicular orientation relative to a longitudinal axis of the LAA 150. The membrane 104 or a proximal surface of the membrane 104 is positioned to prevent the passage of embolic or other material to or from the LAA 150.

In the implanted and deployed state, the frame 101, and particularly the support structure 110 associated with the frame 101 and/or membrane 104, can experience forces placed on it by the subject's heart. Certain embodiments disclosed herein provide for the utilization of deflection activity of the frame 101 and/or membrane 104 (e.g., the support structure 110) for power generation, wherein such power can be used to power one or more sensors 120 and/or circuitry 130. For example, piezoelectric elements can be associated with the frame 101 and/or membrane 104 (e.g., the support structure 110) such that pressure and/or strain on the frame 101 and/or membrane 104 can cause corresponding pressure and/or strain on the piezoelectric element(s). By straining the piezoelectric elements (e.g., through direct piezoelectric effect), the movement or deformation of the frame 101 and/or membrane 104 can generate charge on the surface of the piezoelectric polymer. The resulting capacitive buildup in the polymer can provide a voltage source that can be used to, for example, trickle-charge a battery, which can be part of the circuitry 130 or disposed at a separate location, to power various devices, such as blood pressure sensors, blood glucose meters, pacemakers, and/or other sensors 120.

One or more of the sensors 120 can be positioned on an outer surface of the membrane 104 so that, in the implanted and deployed state, the sensors 120 are exposed to blood flow through the heart. The circuitry 130 (including a battery and an antenna, in some embodiments) can be advantageously housed in the cavity created by the frame 101 and the membrane 104.

FIGS. 1E and 1F illustrate example configurations of support structures with piezoelectric material for energy harvesting. FIG. 1E illustrates a top view of an example support structure 110a for use in a percutaneous occluder 100. The occluder 100 with the support structure 110a can be configured for delivery and implantation using a catheter, as described with reference to FIGS. 1C and 1D for example. The configuration of the support structure 110a allows the support structure 110a to be folded or otherwise compacted for delivery with a catheter. FIG. 1F illustrates a top view of an example support structure 110b for use in a surgical occluder 100. The occluder 100 with the support structure 110b can be configured for implantation using a surgical method, for example.

The support structures 110a, 110b can be part of the frame 101 and/or the membrane 104. In certain implementations, the frame 101 includes the piezoelectric material of the support structure (e.g., the support structure 110a) in an occluder 100 configured for percutaneous delivery. In some implementations, the membrane 104 includes the piezoelectric material of the support structure 110 (e.g., the support structure 110b) in an occluder 100 configured for surgical delivery.

There are a number of advantages provided by the occluder 100 relative to other implants that incorporate sensors. For example, because of the relatively large volume provided within the occluder 100 for the circuitry 130, less miniaturization is required making the device easier and cheaper to produce. In addition, the relatively large volume allows for more circuitry and sensors to be included, enhancing the sensing and monitoring capabilities of the occluder 100. The larger size of the occluder 100, relative to other implants, also allows for greater energy harvesting capabilities. The occluder 100 can also include an absolute pressure sensor as one of the sensors 120 which is preferable to differential pressure sensors included with other sensor-enabled implants. Furthermore, the relatively large size of the occluder 100 allows for larger sensors to be included with the sensors 120. In some embodiments, the occluder 100 can include electrodes to provide shocks in the case of atrial fibrillation.

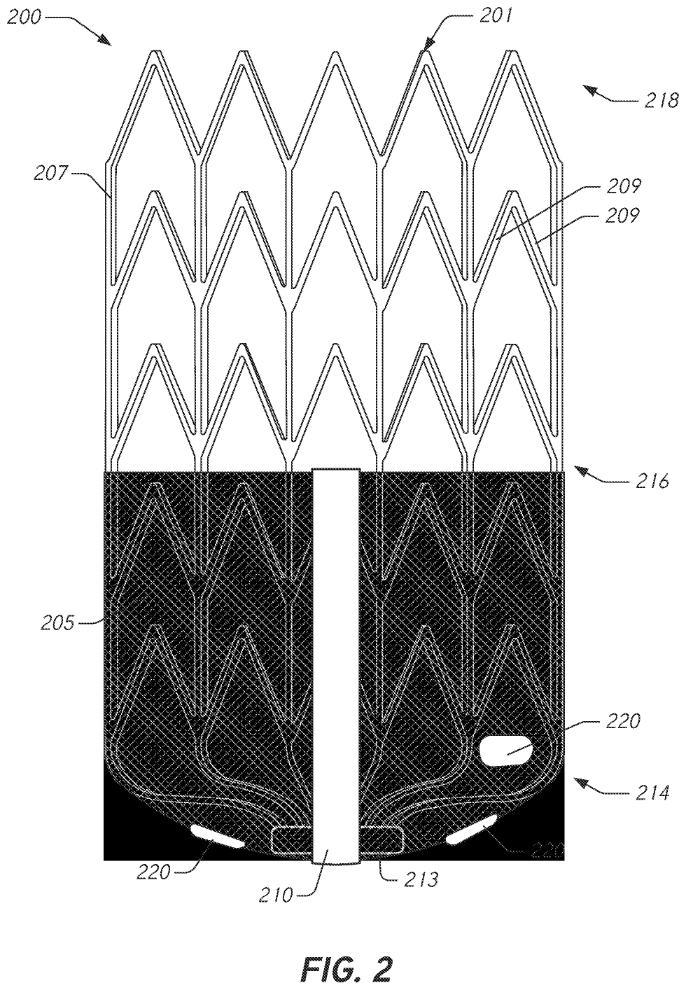

FIG. 2 illustrates an example occlusion device 200 that includes self-powered sensors 220 that receive power from energy harvested by a support structure 210. The occlusion device 200 can be the same or similar to the occluder 100 described above. The occlusion device 200 includes a framework 201 and a biocompatible covering 205 disposed over at least a part of the framework 201. The occlusion device 200 is configured to have sufficient circumferential and/or radial strength to form a seal with the wall of the LAA or other cavity (and to resist the distortive forces that the LAA or cavity may exert on the occlusion device 200) in order to, for example, prevent emboli or blood clots from passing back into the blood stream.

The occlusion device 200 includes the framework 201 that may be formed from, for example, a sheet. The framework 201 may be suitable for use as a component of the occlusion device 200, which may also include the covering 205 (e.g., a filter graft, membrane, etc.). Such a covering 205 can be supported by the framework 201 (e.g., the covering can extend over and from the proximal end of the framework toward the distal end of the framework). The occlusion device 200 (including the framework 201 and covering 205) includes other components, such as the sensors 220 and circuitry (not shown). The occlusion device 200 can be combined with a delivery system for delivering the occlusion device 200 to the LAA or other cavity or body lumen (examples of which are described herein with reference to FIGS. 1C and 1D).

The framework 201 includes a proximal portion 214, a middle portion 216, and a distal portion 218. In some embodiments, the proximal portion 214 includes a hub 213 that has a first diameter. In some embodiments, the middle portion 216 may have a second diameter and can include a plurality of beams 207 extending from the hub 213 to the distal portion 218 that has a third diameter. Each of the plurality of beams 207 may be connected to an adjacent beam 207 by a circumferentially extending column of strut pairs 209. In some embodiments, the framework 201 evenly controls the stability of the longitudinally extending beams 207 by providing a supportive but flexible column of strut pairs 209 between each beam 207 (e.g., support beam).

In some embodiments, each of the plurality of beams 207 is connected to an adjacent beam by a second circumferentially extending column of strut pairs 209. Each beam 207 can include, among other things, a first segment extending from the first hub to the first circumferentially extending column of strut pairs and a second segment extending from the first circumferentially extending column of strut pairs to the second circumferentially extending column of strut pairs. In some embodiments, a strut pair 209 may or may not have the same length as another strut pair 209.

The sensors 220 can be affixed to an outer surface of the covering 205. Circuitry (not shown) can be housed within the volume provided by the framework 201 and the covering 205. For example, circuitry can be affixed to a portion of the framework 201 in an interior portion of the framework 201.

As described elsewhere herein, the support structure 210 can be configured to harvest energy (e.g., as described with reference to the support structure 110). In some embodiments, the support structure 210 is interior to the covering 205. In certain embodiments, the support structure 210 is hosted by the covering 205. In various embodiments, the support structure 210 is integrated into a beam 207 or a plurality of the beams 207 and/or in the struts 209.

### Wireless Monitoring Systems

As described in detail above, patients who receive an occluder or LAA occluder may experience complications after leaving the hospital or extended care facility. These arising complications may require reentry of the patient into the care system, potentially adding significant cost to the overall patient treatment. Disclosed herein are patient monitoring devices and systems, such as including an occlusion device with integrated sensor and wireless communication technology, that allow for the communication of critical patient issues from an implanted device to one or more external devices or systems that can be utilized by care givers and/or patients in the treatment of a patient. For example, an occlusion device can incorporate one or more physiological sensors, which can be incorporated with the occluder or LAA occluder, or otherwise associated therewith.

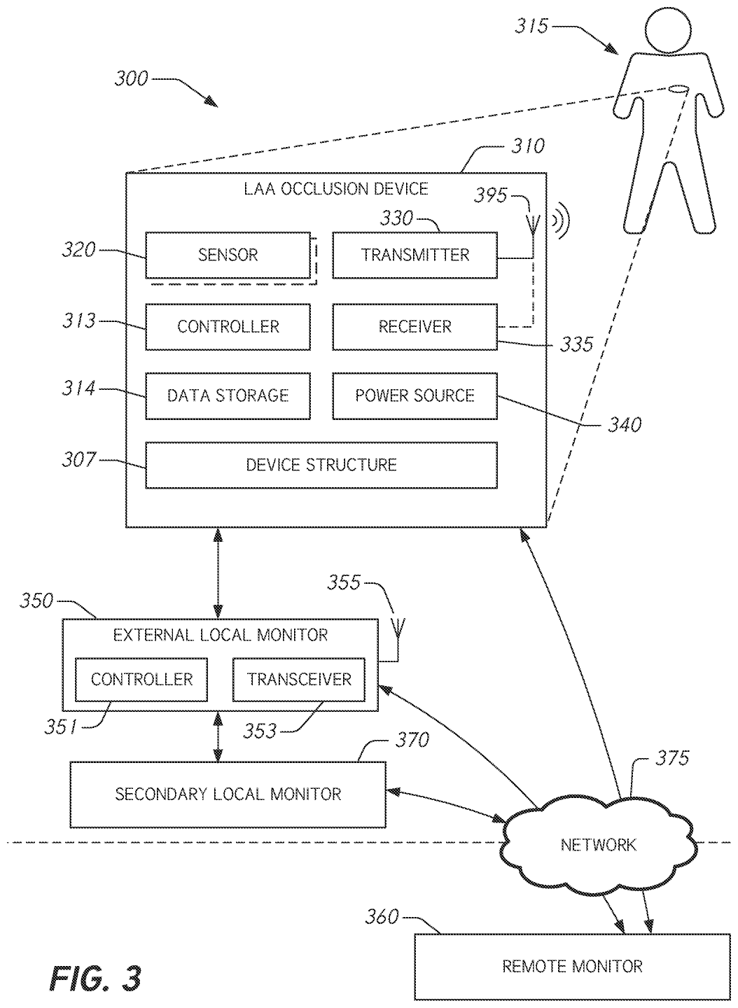

FIG. 3 illustrates a system 300 for monitoring the on-going health of a patient 315 according to one or more embodiments. The patient 315 can have an occlusion device 310 implanted in the LAA or other cavity (not shown) of the patient. For example, the occlusion device 310 can be a prosthetic heart valve, such as an aortic heart valve, as described in detail herein. The occlusion device 310 can include one or more sensor devices 320. The sensor devices 320 can be, for example, one or more microelectromechanical system (MEMS) devices, such as MEMS pressure sensors, or the like.

In certain embodiments, the monitoring system 300 can include at least two sub-systems, including an implantable internal sub-system that includes an occlusion device 310 integrated with one or more physiological parameter sensors 320 (e.g., MEMS pressure sensor(s)), as well as one or more microcontroller(s), discrete electronic component(s), and power and/or data transmitter(s) (e.g., antennae coil). The monitoring system 300 can further include an external (e.g., non-implantable) sub-system that includes a matching external receiver (e.g., coil) electrically and/or communicatively coupled to a patient or physician controller or monitor device. In certain embodiments, both the internal and external sub-systems include a corresponding coil antenna for wireless communication and/or power delivery through patient tissue disposed therebetween. The occlusion device 310 can be any type of occlusion device, examples of which are described herein.

Certain details of the occlusion device 310 are illustrated in the enlarged block 310. The occlusion device 310 can comprise structural features or components 307 as described herein. For example, the device structure 307 can include one or more frames, struts, beams, support structures, coverings, domes, membranes, and/or the like, such as may be consistent with an occluder or occlusion device as described herein. In certain embodiments, one or more of the other components of the occlusion device 310 are integrated with the physical structure 307 of the occlusion device 310. For example, one or more antennas, transmission lines, coils, wires, or the like can be integrated with a structure of the occlusion device, such as a framework of the device 310.

Although certain components are illustrated in FIG. 3 as part of the occlusion device 310, it should be understood that the occlusion device 310 may only comprise a subset of the illustrated components, and can comprise additional components not illustrated. The occlusion device 310 can be the same or similar to the occluder 100 and/or the occlusion device 200. The occlusion device 310 includes one or more sensors 320, which can be configured to provide a response indicative of one or more physiological parameters of the patient 315, such as one or more parameters associated with the function of the occlusion device 310 and the associated organ of the patient 315 (e.g., heart). The sensor(s) 320 can comprise any suitable or desirable sensor(s) for providing signals relating to physiological parameters or conditions associated with the occlusion device 310. In view of the integrated sensor(s) 320, the occlusion device 310 can advantageously provide sensor capability without the need of a separate, stand-alone device that requires a separate procedure to implant.

In certain embodiments, the sensor(s) 320 includes a pressure sensor, such as a pulmonary artery pressure (PAP) measurement device. The sensor(s) 320 can additionally or alternatively include one or more optical sensors, piezoelectric sensors, electromagnetic sensors, strain sensors/gauges, accelerometers, gyroscopes, and/or other types of sensors, which can be positioned in the patient 315 to sense one or more parameters relevant to the function of the occlusion device 310. Sensor signals can be used to track arrhythmia, blood pressure, cardiac output (e.g., as measured by an echo sensor), induction or ballistocardiogram. In certain embodiments, the sensor(s) 320 includes a MEMS pressure sensor, which can be either capacitive or piezoresistive in nature, wherein the sensor is coupled with an application-specific integrated circuit (ASIC) microcontroller. The sensor(s) 320 can be attached to a polyimide flexible circuit substrate and can be further accompanied with one or more discrete electronic components, such as tuning capacitors or the like. In certain embodiments, the sensor(s) 320 includes one or more electrodes for detecting electrical impulses originating in the heart.

In certain embodiments, the sensor(s) 320 can be configured to generate electrical signals that can be wirelessly transmitted to a box/device outside the patient's body, such as the illustrated external local monitor 350. In order to perform such wireless data transmission, the occlusion device 310 can include radio frequency (RF) transmission circuitry, such as a transmitter 330 including an antenna 395. The antenna 395 can comprise an internal antenna coil implanted within the patient. The transmitter 330 can comprise any type of transducer configured to radiate or transmit an electromagnetic signal, such as a conductive wire, coil, plate, or the like. With respect to embodiments that include pressure sensor(s), the voltage change due to the changes in the pressure sensitive element(s) (e.g., capacitance) can be at least somewhat attenuated due to variability in inductive coupling between the occlusion device 310 and a coupled external antenna 355. Such signal attenuation can at least partially limit the placement of the sensor(s) 320 to locations associated with relatively less intense or frequent physiological movement.

The wireless signals generated by the occlusion device 310 can be received by the local external monitor device or subsystem 350, which can include a transceiver module 353 configured to receive the wireless signal transmissions from the occlusion device 310, which is disposed at least partially within the patient 315. The external local monitor 350 can receive the wireless signal transmissions and/or provide wireless power using the external antenna 355, such as a coil. The transceiver 353 can include RF front-end circuitry configured to receive and amplify the signals from the sensor(s) 320, wherein such circuitry can include one or more filters (e.g., band-pass filters), amplifiers (e.g., low-noise amplifiers), analog-to-digital converters (ADC) and/or digital control interface circuitry, phase-locked loop (PLL) circuitry, signal mixers, or the like. The transceiver 353 can further be configured to transmit signals over a network 375 to a remote monitor 360. The RF circuitry of the transceiver 353 can further include one or more of digital-to-analog converter (DAC) circuitry, power amplifier(s), low-pass filters, antenna switch modules, antennas or the like for treatment/processing of transmitted signals over the network 375 and/or for receiving signals from the occlusion device 310. In certain embodiments, the external local monitor 350 includes controller circuitry 351 for performing processing of the signals received from the occlusion device and/or controlling operation of the RF circuitry. The local monitor 350 can be configured to communicate with the network 375 according to a known network protocol, such as Ethernet, Wi-Fi, or the like. In certain embodiments, the external local monitor 350 is a smartphone, laptop computer, or other mobile computing device, or any other type of computing device.

The occlusion device 310 can include controller circuitry 313, which can comprise, for example, one or more chips or dies configured to perform some amount of processing on signals generated and/or transmitted using the device 310. However, due to size, cost, and/or other constraints, the occlusion device 310 may not include independent processing capability in some embodiments.

In certain embodiments, the occlusion device 310 includes a data storage module 314, which can include volatile and/or non-volatile data storage. For example, the data storage module 314 can include solid-state memory utilizing an array of floating-gate transistors, or the like. The controller circuitry 313 can utilize the data storage module 314 for storing sensed data collected over a period of time, wherein the stored data can be transmitted periodically to the external local monitor 350 or other external subsystem. In certain embodiments, the occlusion device 310 does not include any data storage. As described above, the occlusion device 310 is configured with transmitter circuitry 330 for the purpose of wirelessly transmitting data generated by the sensor(s) 320, or other data associated therewith. The occlusion device 310 can further comprise receiver circuitry 335, for receiving input from one or more external subsystems, such as from the external local monitor 350, or from a remote monitor 360 over, for example, the network 375. For example, the occlusion device 310 can receive signals that at least partially control operation of the occlusion device 310, such as by activating/deactivating one or more components or sensors, or otherwise affecting operation or performance of the occlusion device 310.

The one or more components of the occlusion device 310 can be powered by one or more power sources 340. In certain embodiments, the power source 340 can be configured to harvest energy from environmental sources, such as fluid flow, motion, or the like. Due to size, cost and/or electrical complexity concerns, it may be desirable for the power source 340 to be relatively minimalistic in nature. In certain embodiments, the power source 340 is at least partially passive in nature, such that power can be received from an external source wirelessly by passive circuitry of the occlusion device 310, such as through the use of ultrasound, short-range radio frequency transmission, near-field wireless power transmission, or other electromagnetic coupling mechanism. For example, the local monitor 350 can serve as an initiator that actively generates an RF field that can provide power to the occlusion device 310, thereby allowing the power circuitry of the occlusion device to take a relatively simple form factor. Additionally or alternatively, the power source 340 can include a battery, which can advantageously be configured to provide enough power as needed over the monitoring period (e.g., 30, 60, or 90 days, or other period of time).

The external local monitor 350 can serve as an intermediate communication device between the occlusion device 310 and the remote monitor 360. The external local monitor 350 can be a dedicated external unit designed to communicate with the occlusion device 310. For example, the external local monitor 350 can be a wearable communication device, or other device that can be readily disposed in proximity to the patient 315 and occlusion device 310. The external local monitor 350 can be configured to continuously, periodically or intermittently interrogate the occlusion device 310 to extract or request sensor-based information therefrom. In certain embodiments, the external local monitor 350 comprises a user interface, wherein a user can utilize the interface to view sensor data, request sensor data, or otherwise interact with the external local monitor 350 and/or the occlusion device 310.

The system 300 can include a secondary local monitor 370, which can be, for example, a desktop computer or other computing device configured to provide a monitoring station or interface for viewing and/or interacting with the monitor data. In an embodiment, the external local monitor 350 can be a wearable device or other device or system configured to be disposed in close physical proximity to the patient 315 and/or the occlusion device 310, wherein the external local monitor 350 is primarily designed to receive/transmit signals to and/or from the occlusion device 310 and to provide such signals to the secondary local monitor 370 for viewing, processing, and/or manipulation thereof.

The remote monitor subsystem 360 can be any type of computing device or collection of computing devices configured to receive, process and/or present monitor data received over the network 375 from the external local monitor 350, secondary local monitor 370, or occlusion device 310. For example, the remote monitor 360 can advantageously be operated and/or controlled by a healthcare entity, such as a hospital, doctor, or other care entity associated with the patient 315. Although certain embodiments disclosed herein describe communication with the remote monitor 360 from the occlusion device 310 indirectly through the external local monitor 350, in certain embodiments, the occlusion device 310 can comprise a transmitter 330 capable of communicating over the network 375 with the remote monitor subsystem 360 without the necessity of relaying information through the local monitor device 350.

In some embodiments, the occlusion device 310 includes RFID technology for the passive transmission of data. For example, RFID tags can be used to allow a scanner to acquire information from the sensors 320 and/or other components of the occlusion device 310 using passive means. In some embodiments, the occlusion device 310 is configured to transmit blindly without receiving or being capable of receiving information from the external local monitor 350. In some embodiments, the occlusion device 310 can be configured to receive commands from one or more external systems and respond by changing operating parameters or properties of the sensors 320 or controller 313, and/or by transmitting data from the data storage 314 and/or the sensors 320.

### Electronic Sensor Modules

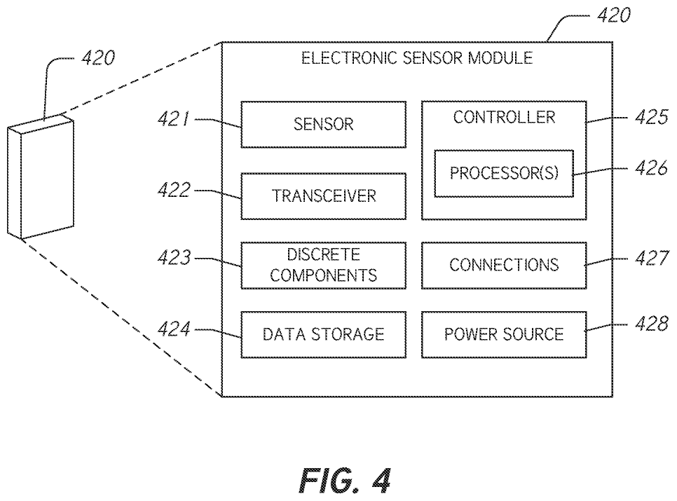

FIG. 4 illustrates a diagram of an electronic sensor module 420 according to one or more embodiments disclosed herein. The sensor module 420 can be any sensor positioned on or included as part of an occluder or LAA occluder, such as the sensors 120, 220, or 320 described herein with reference to FIGS. 1A, 1B, 2A, 2B, and 3. The sensor module 420 can take the form of a microchip (e.g., Application-Specific Integrated Circuit (ASIC)) having one or more electrical devices or components housed within an exterior housing, which can be rectangular or have any other shape. The sensor module 420 can include a controller 425 having one or more processors 426 to control operation of components of the sensor module 420. The sensor module 420 can include data storage 424 to store calibration data, measurement data, metadata, executable instructions, and the like. In some embodiments, one or more of the components of the sensor module 420 can be incorporated into circuitry of an occluder or LAA occluder device, as disclosed herein, and electrical connections 427 can be used to transfer data and/or power to and/or from the occluder circuitry.

In certain embodiments, the sensor device 420 can comprise a MEMS pressure sensor that is configured to be exposed to blood flow proximal to a valve implant and sense pressure variations associated with the change in flow velocity. For example, according to Bernoulli's principle, an increase in the speed of a fluid can occur simultaneously with a decrease in pressure. Therefore, for a MEMS pressure sensor device, the varying fluid pressure of the blood flow in contact therewith can cause the membrane/diaphragm element of the pressure chamber/cavity of the MEMS pressure sensor to deflect by some amount.

In some embodiments, the sensor module 420, and/or one or more components thereof, can be coated with a biocompatible protective coating, such as a silver ion coating, or the like. However, certain coatings may interfere with radio-frequency transmission signals and/or electrical circuitry and may therefore be undesirable in some implementations.

In certain embodiments, the sensor module 420 and/or controller 425 associated therewith can be fabricated at least in part using complementary metal-oxide-semiconductor (CMOS) photolithography processes. Suitable substrate materials for the sensor can include silicon dioxide (SiO₂), silicon nitride (e.g., Si₃N₄), sapphire, glass, polyimide, or the like. Suitable materials for metallization and/or interconnect wire bonding can include platinum (Pt), platinum iridium (Pt/Ir), gold (Au), or the like.

The sensor module 420 can include a covering or housing providing biocompatibility and/or increased protection of internal sensor elements or circuitry and/or discrete component(s). For example, the housing or cover can include one or more of silicone, CVD p-xylylene polymer (Parylene), fluorocarbons (e.g., FEP, FTPE, etc.), hydrophilic or hydrophobic coatings, or ceramic coatings such as alumina, zirconia, DLC, ultrananocrystaline diamond, or combinations thereof, which can be applied as coatings or physical structural components.

The controller 425 and/or transceiver 422 can receive the sensor signal from the sensor 421 (e.g., through electrical connections 427) and perform preliminary signal processing and/or digitization. For example, the sensor(s) 421 can provide a voltage differential analog signal (e.g., generated by a MEMS pressure sensor or electrode). The sensor module 420 can further comprise one or more other discrete electrical components 423, such as tuning capacitors or the like, and/or one or more amplifiers (e.g., low-noise amplifier(s)). The substrate (e.g., polyimide) holding the sensor(s), control circuitry, discrete components, and/or other component(s) of the module 420 can be further attached to certain physical structural components of the occlusion device, such as a stent portion of a valve implant along either the inner surface of the orifice, or the outer surface of the valve.

The electronic sensor module 420 can be coupled to an antenna (not shown), such as a coiled antenna, which can be connected to, for example, the substrate and attached to the sewing ring portion of the valve near the inflow aspect of the valve. Suitable material for the coil antennae can be gold (Au), platinum (Pt), platinum iridium (Pt/Ir), or the like. Such materials can provide relatively soft/ductile coil wiring. In certain embodiments, a composite wire with a core made of more rigid material, such as nickel-cobalt alloy (e.g., MP35N alloy, Fort Wayne Metals), cobalt-chromium alloy (e.g., Elgiloy alloy, Elgiloy Specialty Metals), or nitinol.

The components of the sensor module 420, such as the sensor(s) 421, controller 425, transceiver 422, discrete component(s) 423, and/or data storage 424 can be powered by a power source 428. The power source 428 can be an energy harvesting component, examples of which are described herein. Similarly, the power source 428 can be an inductively-powered internal coil antennae configured to receive radio frequency (RF) energy from an external source, examples of which are described herein. Moreover, the power source 428 can be a battery. In some embodiments, the battery in such embodiments can be recharged using energy harvested by an occluder or LAA occluder. In certain embodiments, RF induction can be used to provide a means of bi-directional data communication between the controller 425 of the sensor module 420 that is coupled with the physiological parameter sensor(s) 421 and an external controller of an external local monitor device. Discrete electrical component(s) 423, such as, for example, tuning capacitors or the like, can be utilized to assist in achieving resonance in resonant circuits (e.g., L/C circuits) disposed in the transmission path between the sensor(s) 421 and the monitor device/system.

External Data and/or Power Communication Device/System

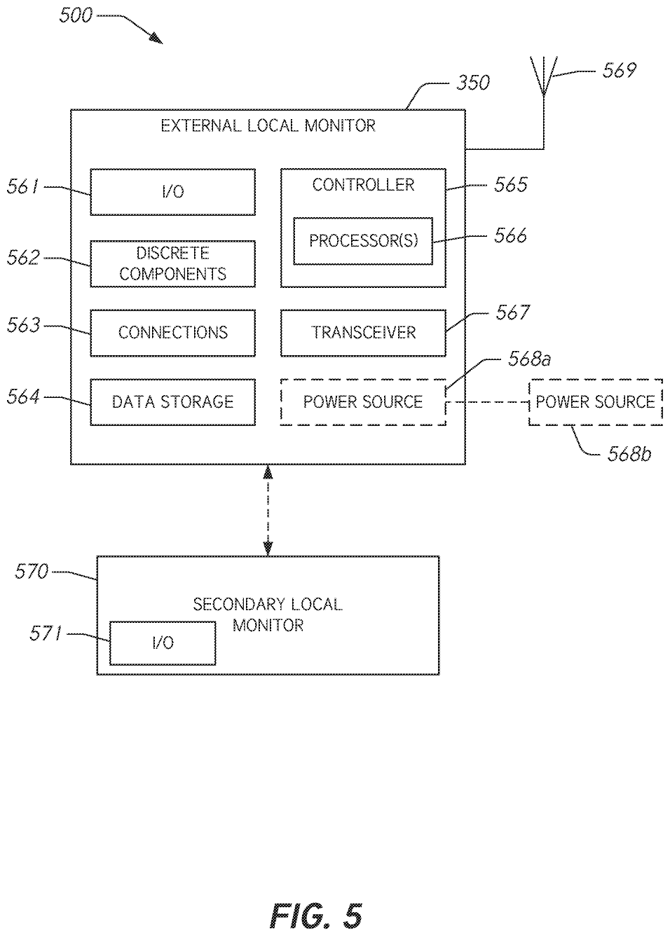

FIG. 5 illustrates a block diagram of an example external local monitor system 500 configured to communicate with a sensor module, such as the sensor module 420 described herein with reference to FIG. 4. Monitoring systems disclosed herein can utilize inductively-coupled transmitters and/or receivers to provide and/or to receive data, power, or both, in communication with an occluder or LAA occluder having one or more integrated physiological parameter sensors. In certain embodiments, digital signals can be transmitted from the sensor modules using radio-frequency (RF) induction, which can provide for signal transfer that is relatively less susceptible to external interference than certain analog solutions may provide.

The external local monitor system 500 can be configured to receive sensor data inductively from a sensor module of an occlusion device (not shown). The external local monitor 350 of the external local monitor system 500 can be configured to receive and/or to process certain metadata, such as device ID or the like, which can also be provided over the data coupling from the implanted sensor module.

The external monitor 350 can comprise a controller 565 with one or more processors 566 and a transceiver 567, which can be communicatively coupled to the implanted sensor module using an antenna 569. In certain embodiments, the antenna 569 can comprise an external coil antenna that is matched and/or tuned to be inductively paired with a corresponding internal coil antenna associated with the internal implant sensor module.

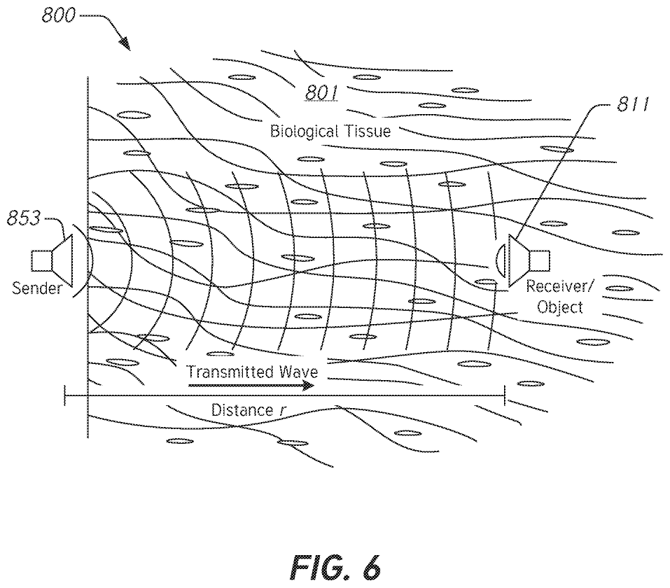

FIG. 6 illustrates a power and/or data communication system 800 system that utilizes ultrasound to transmit power to an occlusion device. The system 800 can be configured to provide wireless ultrasound power charging and/or data communication between an external transmitter module 853 and a receiver module 811, which can be associated with an occlusion device in accordance with the present disclosure and disposed internal to a patient's body, such as in the patient's heart or associated vasculature. Therefore, a certain distance, r, of biological medium 801, including tissue, separates the receiver 811 from the transmitter 853. Because ultrasound communication utilizes mechanical sound waves, in some implementations, the ultrasound transmitter 853 can be configured to generate signals that propagate through the biological medium separating the transmitter 853 and the receiver 811 more efficiently than certain radio-frequency (RF) electromagnetic waves. Therefore, in certain embodiments, power charging using ultrasound transmission in accordance with the system 800 can be more efficient than certain RF power charging implementations. In certain embodiments, the system 800 can be implemented to transmit ultrasound data signals to the receiver 811. Furthermore, in certain embodiments, the receiver 811 can be configured with ultrasound transmission functionality for transmitting data signals (e.g., sensor reading data) to the transmitter 853 or other external module. The ultrasound power and/or data communication system 800 can be particularly useful for embodiments utilizing piezoelectric sensor devices in accordance with embodiments disclosed herein.

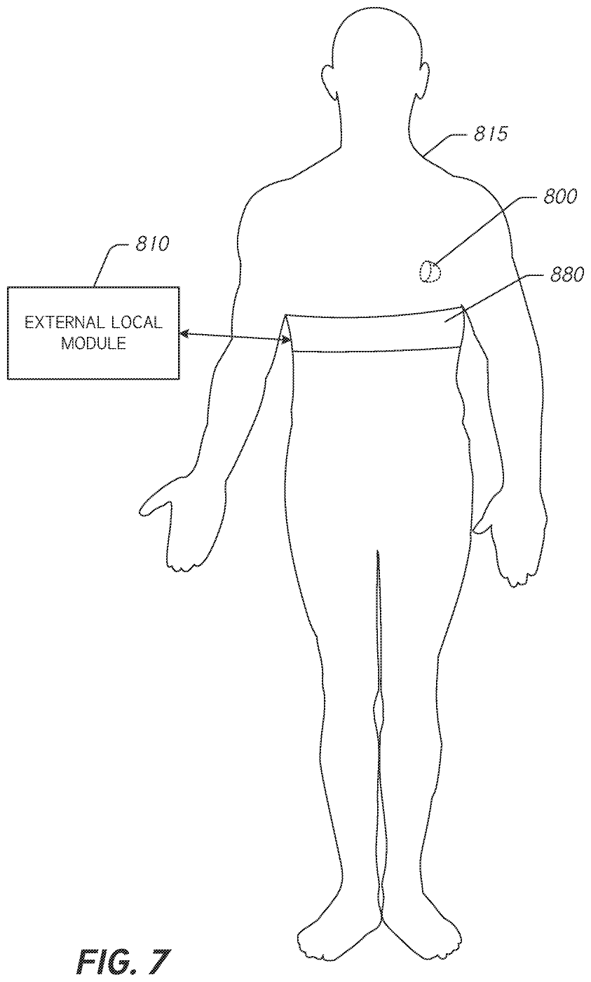

FIG. 7 illustrates an embodiment of an external coil device 880 that can be used for coupling with an occlusion device 800, according to one or more embodiments. The coil device 880 can be configured to be worn on or around the chest and/or torso area of a patient 815, such as underneath the user's armpit, as shown. Such a configuration can allow the external coil device 880 to be relatively close to co-planar with a corresponding internal coil device (e.g., housed within or on the occlusion device 800 implanted in the LAA or another cavity of the patient 815), which can provide desired efficiency with respect to power delivery and/or data communication. The external coil device 880 can be configured to communicate, wired or wirelessly, with an external local module 810 (similar to the external local monitor 350 or secondary local monitor 370, 570 described elsewhere herein).

Returning to FIG. 5, the external local monitor 350 can comprise an integrated power source 568a, such as a battery or other power storage device or element. Alternatively or additionally, the external local monitor 350 can be configured to receive power from an external source 568b, such as a plug-in power source. Use of battery power by the external local monitor 350 can advantageously allow for extended and/or near-continuous monitoring, as well as portability. For example, in certain embodiments, the external local monitor 350 can be carried by the patient, such as on a belt or other wearable article, allowing the patient to carry on daily activities with reduced inconvenience.

The controller 565 can be configured to initialize, calibrate, and/or program the sensor modules. For example, the controller 565 can be configured to program sensor resolution, and/or to adjust data acquisition intervals. During the monitoring period, the controller 565 can be programmed to monitor the sensor modules (e.g., pressure conditions) intermittently, or substantially continuously, and store the monitored data aboard the external monitor 350, such as in the data storage 564, and/or transfer the data to a secondary local monitor 570 for storage and/or use thereby. For example, the secondary local monitor 570 can be a computer to which sensor data can be downloaded once received by the external local monitor 350. The secondary local monitor 570 can be configured to implement more in-depth analysis of the sensor data, possibly in conjunction with cardiopulmonary data acquired from other sources. In certain embodiments, the secondary local monitor 570 can provide 571 input/output (I/O) capability for interaction with the patient or health care provider. For example, the secondary local monitor 570 can comprise a tablet, laptop, desktop, smartphone, or wearable computing device, which can include a visual display as well as user input means, such as a keyboard, touchscreen, or the like. The external local monitor 350 can be coupled to the secondary local monitor 570 over a wired or wireless connection, using an input/output module 561. The external local monitor 350 can include discrete components 562 for processing electrical signals and connections 563 to interconnect components of the external local monitor 350.

### Additional Embodiments and Terminology

The terms “subject” and “patient” are used interchangeably herein and relate to mammals, inclusive of warm-blooded animals (domesticated and non-domesticated animals), and humans. The terms “clinician” and “healthcare provider” are used interchangeably herein.

The term “sensor” as used herein relates to a device, component, or region of a device capable of detecting and/or quantifying and/or qualifying a physiological parameter of a subject. The phrase “system” as used herein relates to a device having components, or to a combination of devices, operating at least in part in a cooperative manner. Sensors generally include those that continually measure the physiological parameter without user initiation and/or interaction (“continuous sensing device” or “continuous sensor”). Continuous sensors include devices and monitoring processes wherein data gaps can and/or do exist, for example, when a continuous pressure sensor is temporarily not providing data, monitoring, or detecting. Sensors also generally include those that intermittently measure the physiological parameter with or without user initiation and/or interaction (“intermittent sensing device” or “intermittent sensor”). In some embodiments, sensors, continuous sensing devices, and/or intermittent sensing devices relate to devices, components, or regions of devices capable of detecting and/or quantifying and/or qualifying a physiological hemodynamic parameter of a subject.

The phrases “physiological data,” “physiological parameter,” and/or “hemodynamic parameter” include without limitation, parameters directly or indirectly related to providing or calculating blood pressure (BP), stroke volume (SV), cardiac output (CO), end-diastolic volume, ejection fraction, stroke volume variation (SVV), pulse pressure variation (PPV), systolic pressure variations (SPV), extravascular lung water index (ELWI), pulmonary vascular permeability index (PVPI), global end-diastolic index (GEDI), global ejection fraction (GEF), systolic volume index (SVI), arterial blood pressure (ABP), cardiac index (CI), systemic vascular resistance index (SVRI), peripheral resistance (PR), central venous saturation (ScvO2), and plethysmographic variability index (PVI). Hemodynamic parameters are inclusive of the absolute value of such parameters, a percentage change or variation in the parameters since an event was recorded, and an absolute percentage change within a previous time segment.

The phrases “electronic connection,” “electrical connection,” “electrical contact” as used herein relate to any connection between two electrical conductors known to those in the art. In some embodiments, electrodes are in electrical connection with (e.g., electrically connected to) the electronic circuitry of a device.

The term and phrase “electronics” and “system electronics” as used herein relate to electronics operatively coupled to the sensor and configured to measure, process, receive, and/or transmit data associated with a sensor, and/or electronics configured to communicate with a monitor or a data acquisition device.

The phrases “operatively connected,” “operatively linked,” “operably connected,” and “operably linked” as used herein relate to one or more components linked to one or more other components, such that a function is enabled. The terms can refer to a mechanical connection, an electrical connection, or any connection that allows transmission of signals between the components. For example, one or more transducers can be used to detect pressure and to convert that information into a signal; the signal can then be transmitted to a circuit. In such an example, the transducer is “operably linked” to the electronic circuitry. The terms “operatively connected,” “operatively linked,” “operably connected,” and “operably linked” include wired and wireless connections.

The term and phrase “controller,” “processor” or “processing module,” as used herein relates to components and the like designed to perform arithmetic or logic operations using logic circuitry that responds to and processes basic instructions, for example, instructions that drive a computer and/or perform calculations of numbers or their representation (e.g., binary numbers).

The terms “substantial” and “substantially” as used herein relate to a sufficient amount that provides a desired function. For example, an amount greater than 50 percent, an amount greater than 60 percent, an amount greater than 70 percent, an amount greater than 80 percent, or an amount greater than 90 percent.

Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described herein. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, any and all of the methods, operations, steps, etc. described herein can be performed on a living animal or on a non-living cadaver, cadaver heart, simulator, anthropomorphic ghost, etc. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments. The terms “comprising,” “including,” “having,” “characterized by,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

Reference throughout this specification to “certain embodiments” or “an embodiment” means that a particular feature, structure or characteristic described in connection with the embodiment is included in at least some embodiments. Thus, appearances of the phrases “in some embodiments” or “in an embodiment” in various places throughout this specification are not necessarily all referring to the same embodiment and may refer to one or more of the same or different embodiments. Furthermore, the particular features, structures or characteristics can be combined in any suitable manner, as would be apparent to one of ordinary skill in the art from this disclosure, in one or more embodiments.

It should be appreciated that in the above description of embodiments, various features are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above but should be determined only by a fair reading of the claims that follow.

What is claimed is:

1. A device for occluding a left atrial appendage (LAA) of a subject, the device comprising:
a membrane with an outer surface and an inner surface, the membrane configured to inhibit passage of blood;
an expandable frame at least partially covered by the membrane, the expandable frame configured to support the membrane in the LAA to substantially occlude the LAA;
a support structure coupled to the membrane or to the expandable frame, the support structure configured to harvest energy from environmental sources within the LAA; and
a plurality of physiological sensors coupled to the outer surface of the membrane, the plurality of physiological sensors configured to receive power from the energy harvested by the support structure.

2. The device of claim 1, further comprising circuitry with electrical connections to the plurality of physiological sensors.

3. The device of claim 2, further comprising an antenna coupled to the circuitry wherein the circuitry includes a transmitter coupled to the antenna to transmit data acquired by one or more of the plurality of physiological sensors.

4. The device of claim 2, wherein the circuitry receives power from the energy harvested by the support structure.

5. The device of claim 4, wherein the circuitry includes a battery that is recharged using the received power.

6. The device of claim 1, wherein the support structure includes a stack of piezoelectric polymers configured to generate electrical power from mechanical deflections or deformations.

7. The device of claim 1, wherein the support structure comprises layers of piezoelectric material separated by conductive plates.

8. The device of claim 1, wherein the support structure is incorporated into the expandable frame.

9. The device of claim 1, wherein the support structure further generates data related to blood pressure.

10. The device of claim 1, wherein the plurality of physiological sensors includes an absolute pressure measurement sensor.

11. The device of claim 1, further comprising an ultrasound receiver module configured to receive ultrasound transmissions.

12. The device of claim 11, wherein the ultrasound receiver module is configured to receive power from an external ultrasound source using ultrasound.

13. The device of claim 12, further comprising an ultrasound transmission module configured to transmit data to the external ultrasound source using ultrasound.

14. The device of claim 1, wherein the expandable frame includes a plurality of longitudinally extending beams coupled together using pairs of struts.

15. The device of claim 1, wherein the support structure is affixed to the outer surface of the membrane.

16. The device of claim 15 wherein the membrane forms a dome structure and the support structure extends over a center of the dome structure.

17. A patient monitoring system comprising:
a left atrial appendage (LAA) occlusion device with a membrane and an expandable frame configured to occlude an LAA of a subject, the membrane including a plurality of sensors that receive electrical power from a power generator associated with the expandable frame or with the membrane, the power generator configured to generate electrical power in response to deformation of the expandable frame or of the membrane, the LAA occlusion device further including an antenna in communication with the plurality of sensors to transmit data acquired with the plurality of sensors; and
an external local monitor configured to receive data transmitted from the LAA occlusion device, the external local monitor including a data display configured to display data acquired with the plurality of sensors of the LAA occlusion device.

18. The system of claim 17 wherein the LAA occlusion device further includes a receiver to receive wireless transmission from the external local monitor.

19. The system of claim 17, further comprising a remote monitor configured to receive data from the external local monitor to enable monitoring of data acquired with the plurality of sensors remotely.

20. The system of claim 17, further comprising a secondary local monitor configured to provide an interface for interacting with the data from the plurality of sensors of the LAA occlusion device.
